(19) United States
(12) Patent Application Publication
(10) Pub. No.: US 2021/0008333 A1
(43) Pub. Date: Jan. 14, 2021

(54) SLEEP INTERVENTION EQUIPMENT, SYSTEM AND METHOD

(71) Applicant: BOE Technology Group Co., Ltd., Beijing (CN)

(72) Inventors: Yao CHEN, Beijing (CN); Yi LI, Beijing (CN); Bao FU, Beijing (CN); Feng ZHANG, Beijing (CN)

(21) Appl. No.: 16/916,505

(22) Filed: Jun. 30, 2020

(30) Foreign Application Priority Data
Jul. 11, 2019 (CN) 201910626623.2

(51) Int. Cl. A61M 21/02; G16H 40/67; G16H 50/30; G16H 20/30; G16H 70/20; G16H 50/70; A61B 5/00; A61N 1/36; A61B 5/05; A61B 5/0205

(52) U.S. Cl. CPC A61M 21/02; G16H 40/67; G16H 50/30; G16H 20/30; G16H 70/20; A61M 2021/0072; A61B 5/4812; A61B 5/4818; A61N 1/36003; A61B 5/05; A61B 5/0205; G16H 50/70

(57) ABSTRACT

A sleep intervention equipment, system and method are provided. The sleep intervention equipment includes a physical sign detection device, a first processor, and a sleep intervention device. The first processor is communicatively connected to the physical sign detection device and the sleep intervention device, respectively. The physical sign detection device is configured to detect a user's physical information without touching the user, and send the physical information to the first processor. The first processor is configured to determine a sleep stage and/or breath state of the user according to the physical information, and send a corresponding control instruction to the sleep intervention device according to the sleep stage and/or breath state of the user. The sleep intervention device is configured to perform a corresponding sleep intervention behavior in response to the control instruction. The sleep stage includes a waking state, an approaching waking state, or a sleep state.

### CROSS-REFERENCE TO RELATED APPLICATIONS

The present application claims the priority from Chinese Patent Application No. 201910626623.2, filed with the Chinese Patent Office on Jul. 11, 2019, the content of which is hereby incorporated herein by reference in its entirety.

### TECHNICAL FIELD

The present disclosure relates to but not limited to the field of sleep intervention technology, particularly relates to a sleep intervention equipment, system and method.

### BACKGROUND

A sleep intervention equipment is used to monitor and intervene a user's sleep, for example, to monitor whether the user has an Obstructive Sleep Apnea Hypopnea Syndrome (OSAS) during sleep. Means commonly used to monitor the user's sleep are, for example, a wristband sleep activity recorder or a professional sleep mattress being used to monitor a weak vibration caused by the user's breathing. A relevant sleep intervention equipment will touch the user's body when monitoring the user's sleep, disturbing the user's sleep and affecting the user's sleep quality.

### SUMMARY

According to an aspect of embodiments of the present disclosure, a sleep intervention equipment is provided, including a physical sign detection device, a first processor, and a sleep intervention device. The first processor is communicatively connected to the physical sign detection device and the sleep intervention device, respectively. The physical sign detection device is configured to detect a user's physical information without touching the user, and send the physical information to the first processor. The first processor is configured to determine a sleep stage and/or breath state of the user according to the physical information, and send a corresponding control instruction to the sleep intervention device according to the sleep stage and/or breath state of the user. The sleep intervention device is configured to perform a corresponding sleep intervention behavior in response to the control instruction. The sleep stage includes a waking state, an approaching waking state, or a sleep state.

In a possible embodiment, the first processor is configured to determine that the user is in a state of non-waking and apnea according to the physical information, and send a first instruction to the sleep intervention device; and the sleep intervention device is configured to perform a contact-type intervention behavior on the user in response to the first instruction.

In a possible embodiment, the first processor is configured to determine that the user is changed from the state of apnea to a state of normal breathing according to the physical information, and send a second instruction to the sleep intervention device; and the sleep intervention device is configured to stop performing the contact-type intervention behavior on the user in response to the second instruction.

In a possible embodiment, the sleep intervention device includes an electrical stimulation device configured to: electrically stimulate a genioglossus muscle of the user to expand to open an airway of the user in response to the first instruction; or stop electrically stimulating the genioglossus muscle of the user in response to the second instruction.

In a possible embodiment, the sleep intervention device includes a vibration device configured to: vibrate to induce the user to lie on a side in response to the first instruction; or stop vibrating in response to the second instruction.

In a possible embodiment, the physical sign detection device includes a biological radar.

In a possible embodiment, the physical information includes respiratory data, body movement data, and/or heart rate data.

In a possible embodiment, the sleep intervention equipment further includes a sleep-aid device. The first processor is configured to determine that the user is in the waking state according to the physical information, and send a third instruction to the sleep-aid device; and the sleep-aid device is configured to perform a sleep-aid behavior in response to the third instruction.

In a possible embodiment, the first processor is configured to determine that the user is in the sleep state according to the physical information, and send a fourth instruction to the sleep-aid device; and the sleep-aid device is configured to stop performing a sleep-aid behavior in response to the fourth instruction.

In a possible embodiment, the sleep-aid behavior includes at least one of playing a first sound, emitting a first light, or displaying a first image.

In a possible embodiment, the first light includes red-orange light.

In a possible embodiment, the first sound includes a white noise.

In a possible embodiment, the first processor is configured to determine that the user enters the awake state from the sleep state according to the physical information, and send a fifth instruction to the sleep-aid device; and the sleep-aid device is configured to perform a wake-up behavior in response to the fifth instruction. The wake-up behavior includes at least one of playing a second sound, emitting a second light, or displaying a second image.

In a possible embodiment, the sleep intervention equipment has a split structure, in which the physical sign detection device, the sleep intervention device, and the sleep-aid device are located at a user side, and the first processor is located at a network side.

In a possible embodiment, the sleep intervention equipment has a split structure, in which the physical sign detection device and the sleep intervention device are located at a user side, and the first processor is located at a network side.

According to another aspect of embodiments of the present disclosure, a sleep intervention system is provided, including the sleep intervention equipment according to the embodiments of the present disclosure, and a sleep intervention management system communicatively connected to the sleep intervention equipment. The sleep intervention management system includes a second processor and a memory. The memory is configured to store intervention behavior plans respectively corresponding to respective sleep stages and/or breath states; and the second processor is configured to obtain the sleep stage and/or breath state of the user from the sleep intervention equipment, acquire a corresponding intervention behavior plan from the memory according to the sleep stage and/or breath state of the user, and feedback the acquired intervention behavior plan to the sleep intervention equipment.

In a possible embodiment, the memory is further configured to store sleep-aid behavior plans respectively corresponding to the respective sleep stages and/or breath states; and the second processor is further configured to acquire a corresponding sleep-aid behavior plan from the memory according to the sleep stage and/or breath state of the user, and feedback the acquired sleep-aid behavior plan to the sleep intervention equipment.

In a possible embodiment, the memory is further configured to store wake-up behavior plans respectively corresponding to the respective sleep stages and/or breath states; and the second processor is further configured to acquire a corresponding wake-up behavior plan from the memory according to the sleep stage and/or breath state of the user, and feedback the acquired wake-up behavior plan to the sleep intervention equipment.

According to still another aspect of embodiments of the present disclosure, a sleep intervention method is provided, the method including: detecting a user's physical information without touching the user; determining a sleep stage and/or breath state of the user according to the physical information; and performing a corresponding sleep intervention behavior in response to the determined sleep stage and/or breath state of the user. The sleep stage includes a waking state, an approaching waking state, or a sleep state.

In a possible embodiment, determining the sleep stage and/or breath state of the user according to the physical information, and performing the corresponding sleep intervention behavior in response to the determined sleep stage and/or breath state of the user, includes: determining that the user is in a state of non-waking and apnea according to the physical information, and performing a contact-type intervention behavior on the user in response to that the user is determined in the state of non-waking and apnea.

In a possible embodiment, determining the sleep stage and/or breath state of the user according to the physical information, and performing the corresponding sleep intervention behavior in response to the determined sleep stage and/or breath state of the user, includes: determining that the user is changed from the state of apnea to a state of normal breathing according to the physical information, and stopping performing the contact-type intervention behavior on the user in response to that the user is determined to be changed from the state of apnea to the state of normal breathing.

In a possible embodiment, performing the contact-type intervention behavior on the user, includes: electrically stimulating a genioglossus muscle of the user to expand to open an airway of the user, and/or vibrating to induce the user to lie on a side.

In a possible embodiment, stopping performing the contact-type intervention behavior on the user, includes: stopping electrically stimulating the genioglossus muscle of the user, and/or stopping vibrating.

In a possible embodiment, the physical information includes respiratory data, body movement data, and/or heart rate data.

In a possible embodiment, the sleep intervention method further includes: determining that the user is in the waking state according to the physical information, and performing a sleep-aid behavior in response to that the user is determined in the waking state.

In a possible embodiment, the sleep intervention method further includes: determining that the user is in the sleep state according to the physical information, and stopping performing the sleep-aid behavior in response to that the user is determined in the sleep state.

In a possible embodiment, the sleep-aid behavior includes at least one of playing a first sound, emitting a first light, or displaying a first image.

In a possible embodiment, the sleep intervention method further includes: determining that the user enters the awake state from the sleep state according to the physical information, and performing a wake-up behavior in response to that the user is determined to enter the awake state from the sleep state. The wake-up behavior includes at least one of playing a second sound, emitting a second light, or displaying a second image.

### BRIEF DESCRIPTION OF THE DRAWINGS

FIG. 1 is a schematic structural diagram of a sleep intervention equipment according to an embodiment of the present disclosure.

FIG. 2 is a schematic structural diagram of a sleep intervention system according to an embodiment of the present disclosure.

FIG. 3 is a schematic flowchart of a sleep intervention method according to an embodiment of the present disclosure.

### DETAILED DESCRIPTION

In order to enable one of ordinary skill in the art to better understand the technical solutions of the present disclosure, the present disclosure will be further described below in details with reference to the accompanying drawings and exemplary embodiments.

As shown in FIG. 1, it is a schematic structural diagram of a sleep intervention equipment according to an embodiment of the present disclosure. The sleep intervention equipment may include a physical sign detection device 1, a processor 2, and a sleep intervention device 3. The processor 2 is communicatively connected to the physical sign detection device 1 and the sleep intervention device 3, respectively.

The physical sign detection device 1 may be configured to detect a user's physical information without touching the user. The processor 2 may be configured to determine a sleep stage and/or breath state of the user according to detection data obtained by the physical sign detection device 1. The sleep intervention device 3 may be configured to perform a contact-type apnea intervention behavior on the user, if the processor 2 determines that the user is in a state of non-waking and apnea.

According to an embodiment of the present disclosure, the physical sign detection device 1 may be a pressure sensor or a radio frequency radar.

In some illustrative examples, the radio frequency radar may be a micro power radio frequency radar of 2.45 GHz microwave.

In some illustrative examples, the radio frequency radar may also be a 24 GHz biological radar.

In some illustrative examples, the radio frequency radar may also be an ultra-wideband (UWB) biological radar.

Radio waves emitted by the radar can penetrate a non-metallic medium, and detect the user's vital signs (i.e., the physical information) without touching the user's body through any electrodes or sensors, thereby reducing interference to the user's sleep.

In some illustrative examples, a UWB biological radar may be used. The U.S. Federal Communications Commission (FCC) defines: a signal with a relative bandwidth greater than 20% or with an absolute bandwidth greater than 500 MHz in a wireless communication system is an ultra-wideband signal.

The UWB biological radar has the following characteristics: very narrow pulse (a pulse width is tens of nanoseconds or dozens of picoseconds, a frequency extends from zero to tens of gigahertz; and the bandwidth for UWB system specified by FCC is 3.1 GHz˜10.6 GHz), no carrier, high data transmission rate, large system capacity, low power consumption, strong resistance to multipath interference, and good electromagnetic compatibility.

In an implementation of the present disclosure, the UWB biological radar used may have a center frequency ranging from 4 GHz to 10 GHz and a narrow pulse width ranging from 1.5 ns to 5 ns.

Although a number of examples of biological radars are listed above, other detection devices are also available, for example, a laser-based or optical imaging-based detection device.

According to an embodiment of the present disclosure, the physical sign detection device 1 may be configured to detect the user's respiratory data, body movement data, and/or heart rate data.

The processor 2 may analyze which sleep stage the user is in, based on the physical information detected by the physical sign detection device 1, for example, deep sleep, light sleep, or a waking state; and analyze whether the user is in apnea.

For example, taking detecting the user's physical information based on a biological radar as an example, the processor 2 demodulates two I/Q baseband quadrature signals of radar echoes by arc tangent demodulation, and separates a respiratory signal, a heart rate signal and a body movement signal according to different frequency characteristics thereof (e.g., a range of a breathing frequency is generally 0.15 Hz to 0.45 Hz, a range of a heart rate is generally 0.83 Hz to 3.3 Hz, and a body movement frequency is generally 3 Hz to 4 Hz).

On the basis of the above, a sleep stage may be determined. Sleep stages may be divided into a waking state (i.e., a waking period), an approaching waking state (i.e., an REM period) and a sleep state (i.e., an NREM period). The NREM period may be divided into a deep sleep state (i.e., a deep sleep period) and a light sleep state (i.e., a light sleep period).

1) A relationship between the sleep stages and the heart rate: the user's heart rate in the NREM period is generally 10 to 30 times/min slower than that in the waking state, and the user's heart rate in the deep sleep state is the lowest and stable. When a duration of the REM period is greater than a certain period of time, for example, the range of the period is 20 min to 30 min, the heart rate usually does not continue to remain at a high level, but rather large fluctuations occur (an oscillation period is about 20 minutes). As sleep stages (i.e., sleep cycles) change, the heart rate usually changes significantly. In a sleep cycle, an amplitude of the heart rate change is relatively small, and a duration of the change may also be short. When entering the REM period from the NREM period, a cardiac cycle declines gently, and falls to a bottom within about 6-10 minutes. A change from the NREM period to the waking state is generally accompanied by a sudden decline in the cardiac cycle (a duration of the decline is generally within 15 seconds).

2) A relationship between the sleep stages and a respiratory cycle: the user's breathing frequency in the NREM period is slow and stable. Especially in the deep sleep period, the breathing is the most stable, a regular reliability is high, and a breathing pattern is regular. The respiratory signal becomes irregular in the REM period, and the breathing frequency changes more rapidly. The breathing frequency changes with the alternation of the NREM period and the REM period. In the deep sleep state, the breathing frequency changes slowly, and a variance of a deep sleep breathing signal characteristic parameter and the number of breathing times becomes smaller. An amplitude of the respiratory signal changes steadily, and an accumulation of differences in the amplitude of the respiratory signal decreases during the deep sleep period.

3) A relationship between the sleep stages and a body movement: an amplitude and frequency of the body movement in the waking state are relatively large; while during the sleep state, occasional rollovers may occur, and body's local movements are more common and usually shorter, with a smaller amplitude and a lower frequency of occurrence. During both of the light sleep state and the REM period, body movements will occur.

According to an embodiment of the present disclosure, based on above characteristics of data, sleep stages may be distinguished through the following steps 1 to 4.

In step 1, according to the characteristics of the data, such as the characteristics that the cardiac cycle and the respiratory cycle during a state of non-waking are longer, and the body movements during the waking state are denser and last longer, the waking state may be distinguished from the state of non-waking.

In step 2, according to the characteristics of the data, such as the characteristics that a change of the cardiac cycle when switching from the NREM period to the REM period is different from that when awakening from the NREM period; and the breathing frequency in the NREM period is slower and smoother, while the respiratory signal becomes irregular and the frequency changes more quickly during the REM period, the NREM period may be distinguished from the REM period.

In step 3, according to the characteristics of the data, such as the characteristics that during the deep sleep state, the heart rate and breathing are slow and steady, the variance of the respiratory signal characteristic parameter and the number of breathing times becomes smaller, the amplitude of the respiratory signal changes steadily, and the accumulation of differences in the amplitude of the respiratory signal decreases, the deep sleep state may be distinguished from the light sleep state.

In step 4, a calibration is performed.

According to the guidelines for the diagnosis and treatment of obstructive sleep apnea hypopnea syndrome, sleep apnea is defined as: the disappearance or significant weakening of the oral and nasal breathing airflow during sleep (e.g., a decrease in amplitude from a baseline is greater than or equal to 90%) and the duration is greater than or equal to 10 seconds. Changes in chest pressure induced by breathing are detected by using an esophageal manometry, and collected signals are compared with signals recorded in the normal breathing state. Compared with the state of normal breathing, an amplitude of chest movements and a pressure in the chest and abdomen are obviously reduced during the state of apnea. As can be seen from above, when apnea occurs, the amplitude of the respiratory signal is significantly smaller than an amplitude of a normal respiratory signal. Therefore, after the respiratory signal is extracted from the radar echo, a respiration amplitude threshold judgment method may be used to determine whether the apnea occurs; that is, when a respiration amplitude drops by more than 90% from the normal baseline, it can be considered that apnea has occurred.

After determining the sleep stage and/or breath state of the user, the processor 2 may transmit an instruction to control the sleep intervention device 3 to intervene in the user's sleep. Especially when it is determined that the user has apnea, timely intervention can not only improve the quality of sleep, but also prevent accidental death.

According to an embodiment of the present disclosure, the sleep intervention device 3 may include an electrical stimulation device 31. The electrical stimulation device 31 may be configured to electrically stimulate a genioglossus muscle of the user to expand to open an airway of the user.

When being used, the electrical stimulation device 31 may be attached to a position of the user's larynx and generate electrical stimulation to the genioglossus muscle of the larynx to stimulate its expansion, thereby opening the airway of the user and inhibiting the occurrence of apnea.

According to an embodiment of the present disclosure, the electrical stimulation device 31 may include an electrical stimulation module and an electrode sheet. The electrical stimulation module may generate electrical pulses and transmit the electrical stimulation to the genioglossus muscle through the electrode sheet to stimulate the expansion of the genioglossus muscle.

According to an embodiment of the present disclosure, the sleep intervention device 3 may include a vibration device 32. The vibration device 32 may be configured to vibrate to induce the user to lie on a side.

When being used, the vibration device 32 may be attached to a back of the user to apply vibration stimulation to the user, thereby inducing the user to change his sleeping posture to a side lying type. Since the pressure on the airway of the user in the sleeping posture lying on the side is reduced, it is advantageous for the user to open the airway.

According to an embodiment of the present disclosure, the vibration device 32 may include a vibration module, a motion sensor (e.g., a three-axis sensor, a six-axis sensor), and a binding elastic band. First, the binding elastic band binds the vibration device 32 and a human body, so that the vibration device 32 is comfortably fixed on a back of the human body. When the processor 2 determines that intervention is needed, the processor 2 controls the vibration module to generate vibration until the human body changes to the sleeping posture lying on the side, and the motion sensor senses posture change information and sends the posture change information to the processor 2, and at the same time, the vibration module stops vibrating.

It can be understand that after the processor 2 determines that the user's apnea has disappeared, it will transmit an instruction to control the sleep intervention device 3 to stop the above intervention.

It can be understand that because the sleep intervention device 3 contacts the user, the sleep intervention device 3 and the processor 2 may be not located in a same packaging structure. In order to realize a wireless communication between the sleep intervention device 3 and the processor 2, the connection between the two components may be realized through Bluetooth, Wi-Fi, Zigbee and so on. For this reason, corresponding wireless communication devices may be arranged on the sleep intervention device 3 and the processor 2 respectively, and connection pairing may be performed.

According to an embodiment of the present disclosure, the sleep intervention equipment may further include a sleep-aid device 4 communicatively connected to the processor 2. The sleep-aid device 4 may be configured to perform a sleep-aid behavior when the processor 2 determines that the user is awake. For example, a sleep-aid sound is played, a sleep-aid light is emitted, and/or a sleep-aid image is displayed, etc.

In other words, the processor 2 may transmit an instruction to the sleep-aid device 4 after determining that the user is awake at preset or user-set sleep time, and control the sleep-aid device 4 to help the user enter sleep.

According to an embodiment of the present disclosure, the sleep-aid light may include red-orange light, for example, light having a wavelength of 550 nm-750 nm. Of course, the sleep-aid light may also be other dim and soft light.

According to an embodiment of the present disclosure, the sleep-aid sound may include a white noise. Of course, the sleep-aid sound may also be light music.

According to an embodiment of the present disclosure, the sleep-aid image may include a calm and comfortable image that helps fall asleep.

According to an embodiment of the present disclosure, the sleep-aid device 4 may be configured to stop performing the sleep-aid behavior when the processor 2 determines that the user is in the sleep state. On one hand, it is more energy-saving; and on the other hand, the interference to the user's sleep may be reduced.

According to an embodiment of the present disclosure, the sleep-aid device 4 may also be configured to perform a wake-up behavior when the processor 2 determines that the user is about to enter the awake state from the sleep state. The wake-up behavior may include playing a wake-up sound, emitting a wake-up light, and/or displaying a wake-up image, etc.

That is, the processor 2 determines that the user is about to wake up, transmits an instruction to the sleep-aid device 4, and the sleep-aid device 4 wakes up the user.

For example, the wake-up sound may be a wake-up music from weak to strong, the wake-up light may be a soft natural light, and the wake-up image may be a lively and natural scenery, so as to wake up the user in a more comfortable way.

It can be understand that, in order to perform the above-mentioned sleep-aid and wake-up functions, the sleep-aid device 4 may include a speaker, a Light Emitting Diode (LED) light, and/or a display screen.

According to an embodiment of the present disclosure, the sleep intervention equipment may have an integrated structure, and each component is located at a local end.

According to an embodiment of the present disclosure, the sleep intervention equipment may have a split structure, the physical sign detection device 1 and the sleep intervention device 3 are located at the local end (i.e., a user side), and the processor 2 is located at a remote end (i.e., a network side).

According to an embodiment of the present disclosure, the sleep intervention equipment may have the split structure, the physical sign detection device 1, the sleep intervention device 3, and the sleep-aid device 4 are located at the local end, and the processor 2 is located at the remote end.

The local end refers to the user side (but not refers to being integrated in a same shell package structure), which is relative to a remote server end on a non-user side, etc. A communication between the local end and the remote end may be achieved through various methods, such as a Wireless Wide Area Network (WWAN) which may be realized through wireless communication networks such as 3G, 4G, and 5G, and/or a wireless metropolitan area network which may be accessed through the World Interoperability for Microwave Access (WiMAX) and other wireless broadbands. Further, the wired or wireless network-based communication between the local end and the remote server may also be achieved by setting up relay devices, such as wireless routers, Zigbee routers and/or Bluetooth nodes. Furthermore, it is also possible to connect the sleep intervention equipment to a mobile phone, a tablet computer, a notebook computer or other devices on the user side through Bluetooth or Wi-Fi, and then use these devices to communicate with the remote server through a wired network or a wireless network.

By separating the processor 2 from the remote server, it is helpful for developers to continuously update relevant algorithms for dividing sleep stages and breath states and update specific technical implementations of intervention behavior plans, sleep-aid behavior plans, and wake-up behavior plans, which may avoid frequently overall updating the sleep intervention equipment, reduce the power consumption of the sleep intervention equipment, and simplify a volume and a structural complexity of the product.

It can be understand that the music, pictures and other resources used for wake-up or sleep-aid may be stored locally, or may be transmitted from the user's mobile phone or tablet; and the sleep intervention equipment may also be provided with a memory to store the corresponding resources.

It can be understand that when the above processor 2 is set at the remote end, in order to manage basic functions of each device, such as power management, power on/off management, etc., the local end may also be provided with a corresponding local processor to perform the required functions. The local processor may be implemented with a Micro Control Unit (MCU), such as a microcontroller or an Advanced RISC Machine/Million Instructions Per Second (ARM/MIPS) processor with a streamlined instruction set structure.

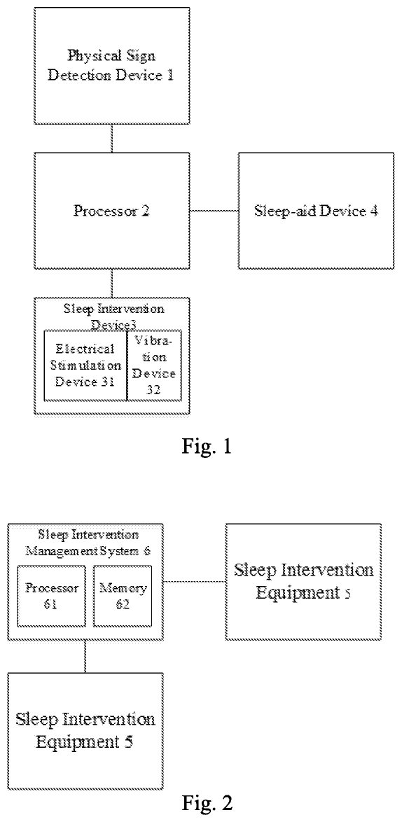

An embodiment of the present disclosure further provides a sleep intervention system, as shown in FIG. 2 which is a schematic structural diagram of a sleep intervention system according to an embodiment of the present disclosure, the sleep intervention system may include at least one above-mentioned sleep intervention equipment 5, and a sleep intervention management system 6 communicatively connected to the sleep intervention equipment 5. The sleep intervention management system 6 may include a processor 61 and a memory 62. The memory 62 may be configured to store intervention behavior plans respectively corresponding to respective sleep stages and/or breath states; and the processor 61 may be configured to obtain the sleep stage and/or breath state of the user from the sleep intervention equipment 5, acquire a corresponding intervention behavior plan from the memory 62 according to the sleep stage and/or breath state of the user, and feedback the acquired intervention behavior plan to the sleep intervention equipment 5.

According to an embodiment of the present disclosure, the memory 62 may further be configured to store sleep-aid behavior plans respectively corresponding to the respective sleep stages and/or breath states; and the processor 61 may further be configured to acquire a corresponding sleep-aid behavior plan from the memory 62 according to the sleep stage and/or breath state of the user, and feedback the acquired sleep-aid behavior plan to the sleep intervention equipment 5.

According to an embodiment of the present disclosure, the memory 62 may further configured to store wake-up behavior plans respectively corresponding to the respective sleep stages and/or breath states; and the processor 61 may further configured to acquire a corresponding wake-up behavior plan from the memory 62 according to the sleep stage and/or breath state of the user, and feedback the acquired wake-up behavior plan to the sleep intervention equipment 5.

It can be understand that, when the sleep intervention equipment 5 is designed as the split structure, the processor 61 of the sleep intervention management system 6 may be integrated with the processor 2 of the sleep intervention equipment 5 into a single processor.

The processor 61 of the sleep intervention management system 6 may be a central processing unit (CPU), a digital signal processor (DSP), an MCU, an application specific integrated circuit (ASIC), or a Field Programmable Gate Array (FPGA), etc. The above-mentioned functions may be realized by executing application programs (e.g., being executed by the CPU) or being programmed to have corresponding functions (e.g., the FPGA) or being solidified to have specific functions (e.g., the ASIC).

The memory 62 of the sleep intervention management system 6 may be a mechanical hard disk (HDD), a solid state disk (SSD), a flash memory, a Secure Digital Memory Card (SD), a Micro SD, a Compact Flash (CF) card, an Embedded Multi Media Card (eMMC) or other storage medias. Correspondence relationships between the respective sleep stages and/or breath states, and the intervention behavior plans, the sleep-aid behavior plan and/or the wake-up behavior plans are stored in the memory 62, which makes it possible to continuously update these correspondence relationships by a management device (e.g., the processor 61 of the sleep intervention management system 6) to better meet the needs of users.

According to an embodiment of the present disclosure, music, pictures, etc. for sleep-aids, wake-up, etc. may be stored in the memory 62 of the sleep intervention management system 6. When the sleep intervention equipment 5 is the used by a user, the user may obtain music, pictures and other resources to be used through network transmission, and these resources may be cached in the memory of the sleep intervention equipment 5. This design makes it possible to continuously add and update related resources in the sleep intervention management system 6 to better meet the personalized needs of users.

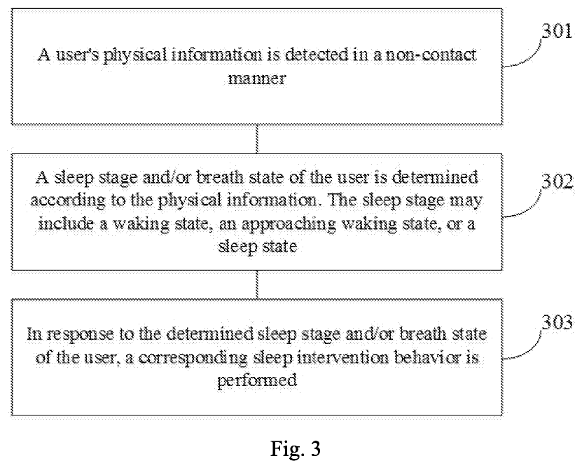

An embodiment of the present disclosure further provides a sleep intervention method, as shown in FIG. 3, it is a schematic flowchart of a sleep intervention method according to an embodiment of the present disclosure. The method may include steps S301 to S303.

In step S301, a user's physical information is detected in a non-contact manner.

In step S302, a sleep stage and/or breath state of the user is determined according to the physical information. The sleep stage may include a waking state, an approaching waking state, or a sleep state.

In step S303, in response to the determined sleep stage and/or breath state of the user, a corresponding sleep intervention behavior is performed.

According to an embodiment of the present disclosure, determining the sleep stage and/or breath state of the user according to the physical information, and performing the corresponding sleep intervention behavior in response to the determined sleep stage and/or breath state of the user, may include: determining that the user is in a state of non-waking and apnea according to the physical information, and performing a contact-type intervention behavior on the user in response to that the user is determined in the state of non-waking and apnea; or determining that the user is changed from a state of apnea to a state of normal breathing according to the physical information, and stopping performing the contact-type intervention behavior on the user in response to that the user is determined to be changed from the state of apnea to the state of normal breathing.

According to an embodiment of the present disclosure, performing the contact-type intervention behavior on the user may include: electrically stimulating a genioglossus muscle of the user to expand to open an airway of the user, and/or vibrating to induce the user to lie on a side.

According to an embodiment of the present disclosure, stopping performing the contact-type intervention behavior on the user may include: stopping electrically stimulating a genioglossus muscle of the user, and/or stopping vibrating.

According to an embodiment of the present disclosure, the physical information may include respiratory data, body movement data, and/or heart rate data.

According to an embodiment of the present disclosure, the sleep intervention method may further include: determining that the user is in the waking state according to the physical information, and performing a sleep-aid behavior in response to that the user is determined in the waking state; or determining that the user is in the sleep state according to the physical information, and stopping performing a sleep-aid behavior in response to that the user is determined in the sleep state. The sleep-aid behavior includes at least one of playing a first sound, emitting a first light, or displaying a first image.

According to an embodiment of the present disclosure, the sleep intervention method may further include: determining that the user enters the awake state from the sleep state according to the physical information, and performing a wake-up behavior in response to that the user is determined to enter the awake state from the sleep state. The wake-up behavior may include at least one of playing a second sound, emitting a second light, or displaying a second image.

Specific implementations and an implementation body of each step of the sleep intervention method, may refer to the above related description of the sleep intervention equipment and the sleep intervention system, and will not be repeated by the embodiments of the present disclosure.

It can be understood that the above embodiments are merely the exemplary embodiments adopted to explain the principle of the present disclosure, but the present disclosure is not limited thereto. One of ordinary skill in the art can make various variations and improvements therein without departing from the spirit and essence of the present disclosure, and these variations and improvements are also considered to be within the protection scope of the present disclosure.

1. A sleep intervention equipment, comprising a physical sign detection device, a first processor, and a sleep intervention device, the first processor being communicatively connected to the physical sign detection device and the sleep intervention device, respectively; wherein
the physical sign detection device is configured to detect a user's physical information without touching the user, and send the physical information to the first processor;
the first processor is configured to determine a sleep stage and/or breath state of the user according to the physical information, and send a corresponding control instruction to the sleep intervention device according to the sleep stage and/or breath state of the user; and
the sleep intervention device is configured to perform a corresponding sleep intervention behavior in response to the control instruction;
wherein the sleep stage comprises a waking state, an approaching waking state, or a sleep state.

2. The sleep intervention equipment according to claim 1, wherein
the first processor is configured to determine that the user is in a state of non-waking and apnea according to the physical information, and send a first instruction to the sleep intervention device; and the sleep intervention device is configured to perform a contact-type intervention behavior on the user in response to the first instruction; or
the first processor is configured to determine that the user is changed from a state of apnea to a state of normal breathing according to the physical information, and send a second instruction to the sleep intervention device; and the sleep intervention device is configured to stop performing the contact-type intervention behavior on the user in response to the second instruction.

3. The sleep intervention equipment according to claim 2, wherein the sleep intervention device comprises an electrical stimulation device configured to:
electrically stimulate a genioglossus muscle of the user to expand to open an airway of the user in response to the first instruction; or
stop electrically stimulating a genioglossus muscle of the user in response to the second instruction.

4. The sleep intervention equipment according to claim 2, wherein the sleep intervention device comprises a vibration device configured to:
vibrate to induce the user to lie on a side in response to the first instruction; or
stop vibrating in response to the second instruction.

5. The sleep intervention equipment according to claim 1, wherein the physical sign detection device comprises a biological radar.

6. The sleep intervention equipment according to claim 1, wherein the physical information comprises respiratory data, body movement data, and/or heart rate data.

7. The sleep intervention equipment according to claim 1, further comprises a sleep-aid device, wherein
the first processor is configured to determine that the user is in the waking state according to the physical information, and send a third instruction to the sleep-aid device; and the sleep-aid device is configured to perform a sleep-aid behavior in response to the third instruction; or
the first processor is configured to determine that the user is in the sleep state according to the physical information, and send a fourth instruction to the sleep-aid device; and the sleep-aid device is configured to stop performing the sleep-aid behavior in response to the fourth instruction;
wherein the sleep-aid behavior comprises at least one of playing a first sound, emitting a first light, or displaying a first image.

8. The sleep intervention equipment according to claim 7, wherein the first light comprises red-orange light.

9. The sleep intervention equipment according to claim 7, wherein the first sound comprises a white noise.

10. The sleep intervention equipment according to claim 7, wherein
the first processor is configured to determine that the user enters the awake state from the sleep state according to the physical information, and send a fifth instruction to the sleep-aid device; and
the sleep-aid device is configured to perform a wake-up behavior in response to the fifth instruction;
wherein the wake-up behavior comprises at least one of playing a second sound, emitting a second light, or displaying a second image.

11. The sleep intervention equipment according to claim 7, wherein the sleep intervention equipment has a split structure, the physical sign detection device, the sleep intervention device, and the sleep-aid device are located at a user side, and the first processor is located at a network side.

12. The sleep intervention equipment according to claim 1, wherein the sleep intervention equipment has a split structure, the physical sign detection device and the sleep intervention device are located at a user side, and the first processor is located at a network side.

13. A sleep intervention system, comprising the sleep intervention equipment according to claim 1, and a sleep intervention management system communicatively connected to the sleep intervention equipment, and comprising a second processor and a memory; wherein
the memory is configured to store intervention behavior plans respectively corresponding to respective sleep stages and/or breath states; and
the second processor is configured to obtain the sleep stage and/or breath state of the user from the sleep intervention equipment, acquire a corresponding intervention behavior plan from the memory according to the sleep stage and/or breath state of the user, and feedback the acquired intervention behavior plan to the sleep intervention equipment.

14. The sleep intervention system according to claim 13, wherein
the memory is further configured to store sleep-aid behavior plans respectively corresponding to the respective sleep stages and/or breath states; and
the second processor is further configured to acquire a corresponding sleep-aid behavior plan from the memory according to the sleep stage and/or breath state of the user, and feedback the acquired sleep-aid behavior plan to the sleep intervention equipment.

15. The sleep intervention system according to claim 13, wherein:
the memory is further configured to store wake-up behavior plans respectively corresponding to the respective sleep stages and/or breath states; and
the second processor is further configured to acquire a corresponding wake-up behavior plan from the memory according to the sleep stage and/or breath state of the user, and feedback the acquired wake-up behavior plan to the sleep intervention equipment.

16. A sleep intervention method, comprising:
detecting a user's physical information without touching the user;
determining a sleep stage and/or breath state of the user according to the physical information; and
performing a corresponding sleep intervention behavior in response to the determined sleep stage and/or breath state of the user;
wherein the sleep stage comprises a waking state, an approaching waking state, or a sleep state.

17. The sleep intervention method according to claim 16, wherein determining the sleep stage and/or breath state of the user according to the physical information, and performing the corresponding sleep intervention behavior in response to the determined sleep stage and/or breath state of the user, comprises:
determining that the user is in a state of non-waking and apnea according to the physical information, and performing a contact-type intervention behavior on the user in response to that the user is determined in the state of non-waking and apnea; or
determining that the user is changed from a state of apnea to a state of normal breathing according to the physical information, and stopping performing the contact-type intervention behavior on the user in response to that the user is determined to be changed from the state of apnea to the state of normal breathing.

18. The sleep intervention method according to claim 17, wherein
performing the contact-type intervention behavior on the user, comprises: electrically stimulating a genioglossus muscle of the user to expand to open an airway of the user, and/or vibrating to induce the user to lie on a side; or
stopping performing the contact-type intervention behavior on the user, comprises: stopping electrically stimulating a genioglossus muscle of the user, and/or stopping vibrating.

19. The sleep intervention method according to claim 16, wherein the physical information comprises respiratory data, body movement data, and/or heart rate data.

20. The sleep intervention method according to claim 16, further comprises:
determining that the user is in the waking state according to the physical information, and performing a sleep-aid behavior in response to that the user is determined in the waking state; or determining that the user is in the sleep state according to the physical information, and stopping performing a sleep-aid behavior in response to that the user is determined in the sleep state; wherein the sleep-aid behavior comprises at least one of playing a first sound, emitting a first light, or displaying a first image; or
determining that the user enters the awake state from the sleep state according to the physical information, and performing a wake-up behavior in response to that the user is determined to enter the awake state from the sleep state; wherein the wake-up behavior comprises at least one of playing a second sound, emitting a second light, or displaying a second image.
